(19) United States
(12) Patent Application Publication
(10) Pub. No.: US 2008/0102511 A1
(43) Pub. Date: May 1, 2008

(54) PROCESS FOR THE JOINT CULTURE OF AN ASSOCIATION OF MICROORGANISM, USING PYRITE (FeS2) AS AND ENERGY SOURCE

(75) Inventors: Pedro Antonio Morales Cerda, La Reina (CL); Leandro Mauricio Padilla Iglesias, Providencia (CL); Ricardo Badilla Ohlbaum, La Reina (CL)

Correspondence Address: MERCHANT & GOULD PC, P.O. BOX 2903, MINNEAPOLIS, MN, 55402-0903, US

(73) Assignee: BIOSIGMA S.A., Colina (CL)

(21) Appl. No.: 11/862,016

(22) Filed: Sep. 26, 2007

(30) Foreign Application Priority Data
Oct. 27, 2006 (CL) 2909-2006

(51) Int. Cl. C12N 1/20

(52) U.S. Cl. 435/252.4

(57) ABSTRACT

The invention publishes a process for the joint culture of an association of microorganisms using pyrite (FeS₂) as an energy source. This invention particularly publishes the use of a pyrite ore as an energy source in the joint culture of an association of Acidithiobacillus ferrooxidans and Acidithiobacillus thiooxidans type isolated microorganisms known as Wenelen DSM 16786 and Licanantay DSM 17318 respectively.

### BACKGROUND OF THE INVENTION

The invention publishes a process for the joint culture of an association of microorganisms, using pyrite (FeS₂) as an energy source. The invention particularly publishes the use of a pyrite ore as an energy source in the joint culture of an association of isolated Acidithiobacillus ferrooxidans and Acidithiobacillus thiooxidans type microorganisms known as Wenelen DSM 16786 and Licanantay DSM 17318 respectively.

### SUMMARY OF THE INVENTION

Artificial or expressly prepared culture mediums, frequently based on high-purity organic and/or inorganic chemical products, are commonly used when culturing microorganisms. The aim of this is usually to control to a maximum the variables associated with microorganism requirements, and to avoid all potential sources of contamination and inhibition of microbial growth.

For instance, laboratory-scale growths of At. ferrooxidans and At. thiooxidans have been described by Silverman, M. P. & Lundgren D. G. 1959, “Studies on the chemoautotrophic iron bacterium ferrobacillus ferroooxidans I. An Improved Medium and a Harvesting Procedure for Securing High Cell Yields” Journal of Bacteriology. 77: 642-647, and by Cook, T. M. 1964. “Growth of Thiobacillus thiooxidans in shaken culture” Journal of Bacteriology. 88: 620-623, respectively.

The previous approach proves to be very appropriate for laboratory-scale, and even sometimes pilot-test-scale microorganism culture, but for economic reasons it may become impractical, especially when dealing with large-scale biomass production. A common solution for this problem consists in using technical-type reagents with which the cost of medium decreases but potential contamination sources increase, and impurities that may inhibit microorganism growth are added.

So, for culturing microorganisms under industrial conditions, technical grade ammonium sulfate and potassium phosphate-based formulations (Hackl et al. U.S. Pat. No. 5,089,412) have been described. To the same effect, in Chilean patent applications CL2731-2004, and CL 2101-2005, culture mediums known as modified 9K (3.0 g/L of (NH₄)₂SO₄, 0.5 g/L of K₂HPO₄, 0.5 g/L of MgSO₄*7H₂O, 0.1 g/L of KCl and 0.1 g/L of Ca(NO₃)₂, 30 g/L of FeSO₄.7H₂O) and 9KS (3.0 g/L of (NH₄)₂SO₄, 0.5 g/L of K₂HPO₄, 0.5 g/L of MgSO₄.7H₂O, 0.1 g/L of KCl, 0.1 g/L of Ca(NO₃)₂, 1% elemental sulfur or other reduced sulfur compounds) are respectively used.

It is a known fact that in microorganism cultures in mediums such as the ones previously mentioned, the final biomass concentration is limited by the substrate concentration used as an energy source and by the growth inhibition exerted by both the substrate mentioned and the products of its metabolism generated during microbial growth [LaCombe, J., Lueking, D. 1990. “Growth and maintenance of Thiobacillus ferrooxidans cells” Applied and Environmental Microbiology. 56: 2801-2806; Nagpal, S. 1997. “A structured model for Thiobacillus ferrooxidans growth on ferrous Iron” Biotechnology and Bioengineering. 53. 310-319].

On the other hand, the type of microorganism obtained depends on the kind of energy source used: iron in the form of Fe²⁺ compounds for iron oxidizing microorganisms, and sulfur compounds—in an oxidizing state −2.0 and +4—for sulfur oxidizing microorganisms.

The above constitutes a limitation for the design of a mixed biomass production process (iron and sulfur oxidizing) because different strains impose different production conditions such as different substrates and pH.

Therefore, when culturing two or more microorganism species is desired, it is an appealing idea to use the same culture medium, or furthermore, even culture the microorganisms together. This way, the number of process stages decreases, operation complexity is simplified, and in some cases it is possible to benefit from the characteristics that are typical of the underlying chemistry.

Iron sulfides, such as pyrite (FeS₂), which are sources of reduced iron and sulfur therefore constitute an interesting alternative for the production of mixed leaching biomass.

The study by Chong, N., Karamanev, D. G., Margaritas, A. 2002. “Effect of particle-particle shearing on the bioleaching of sulfide minerals” Biotechnology and Bioengineering. 80: 349-357, demonstrates at laboratory-scale the growth of microorganisms such as At. ferrooxidans on pyrite as an energy source, obtaining microorganism concentrations of around 10⁸ cells/ml.

Schippers, A., Jozsa, P. G., Sand, W. 1996. “Sulfur chemistry in bacterial leaching of pyrite”. Applied and Environmental Microbiology. 62: 3424-3431, proposes the formation of thiosulfate (S₂O₃²⁻) during the pyrite degradation cycle. This compound may follow a series of abiotic reactions or be used as an energy source by sulfur-oxidizing bacteria, providing a reason to propose the joint culture of iron-oxidizing and thiooxidizing microorganisms on pyrite.

For example, in the study by Bacelar-Nicolau, P. & Jonson, B. 1999. “Leaching of pyrite by acidophilic heterotrophic iron-oxidizing bacteria in pure and mixed cultures”. Applied and Environmental Microbiology. 65: 585-590, the mixed culture of iron-oxidizing and thiooxidizing microorganisms on pyrite is presented.

From the chemical point of view, for the decomposition of pyrite to be used as an energy source by Acidithiobacillus ferrooxidans type microorganisms, the activity of these microorganisms is represented according to the following formula:

FeS₂+6Fe³⁺+3H₂O→7Fe²⁺+S₂O₃²⁻+6H⁺7Fe²⁺+7/4O₂+7H⁺→7Fe³⁺⁺7/2H₂O+At. ferrooxidansFeS₂+7/4O₂+H⁺→Fe³⁺+S₂O₃²⁻+½H₂O+At. ferrooxidans reaction (i)

As it can be observed in reaction (i), one of the products is thiosulfate, which contemplates sulfur in an intermediate oxidation state, and which, according to the following reaction, is useful as an energy source for Acidithiobacillus thiooxidans type microorganisms:

S₂O₃²⁻+H₂O+2O₂→2SO₄²⁻+2H⁺+At. thiooxidans reaction (ii)

Finally, regarding the use of pyrite or materials that contain it, existing studies put forth different approaches, for example in patents WO0136693, WO0071763 and WO2004027100, its use as a source of sulfuric acid is proposed. In document WO0136693 pyrite is associated with leaching systems in which sulfuric acid is not added; in document WO0071763, its use is linked to the replacement of acid when the ore presents a high demand for acid; and in document WO2004027100, it is used to replace part of the acid needed. In other documents such as U.S. Pat. No. 6,110,253, and US2005103162 application, pyrite is used as a mechanism to increase heap temperature, because when it is bio-oxidized it generates heat, which according to these texts, makes practicing bioleaching with thermophilic microorganisms possible.

As far as we know, there is still a lack of lower cost culture mediums which would make large-scale production of microorganisms useful for bio leaching feasible, and we are not aware of processes in which pyrite is actually used as an energy source for the growth of mixed biomass either.

### BRIEF DESCRIPTION OF THE DRAWINGS

FIG. 1: Growth curves of an association of microorganisms on a culture medium with different mixtures of ferrous sulfate and pyrite concentrate (I), according to the description in Example 1, are presented in this figure.

FIG. 2: The batch-mode growth curve of an association of microorganisms in a culture medium modified with the incorporation of pyrite concentrate (II) as described in Example 2, is presented in this figure.

FIG. 3: At. ferrooxidans WENELEN DSM 16786 (black bars) and At. thiooxidans LICANANTAY DSM 17318 (white bars) contents in a biomass propagation bioreactor operated in a continuous way, using a culture medium modified with the incorporation of pyrite concentrate (III), as described in Example 3, are presented in this figure.

### DESCRIPTION OF THE INVENTION

For a better understanding of the processes, the following should be understood:
a) ATCC: “American Type Culture Collection”, American collection of standard microorganism cultures.
b) Ore bioleaching in troughs: A process carried out in a tank with a false bottom, into which the ore is loaded, and flooded with the leaching solution which is circulated through mineral particles in the presence of acidophilic microorganisms, and the copper is extracted dissolved in an acid solution.
c) Ore bioleaching in dumps: Ores that are positioned below the cut-off grade and are extracted from an “open-pit” mining operation, are gathered “run-of-the-mine” or with primary crushing, in creeks with characteristics that are appropriate for controlling solution infiltration, or on surfaces on which a waterproof covering has previously been installed. The leaching solution is irrigated over the surface in the presence of acidophilic microorganisms, and the copper, dissolved in an acid solution, is extracted from the base.
d) Ore bioleaching in heaps: In this process, the ore which has been crushed down to a specific grading is collected on a waterproof surface on a slight slope. The leaching solution is irrigated over the surface in the presence of acidophilic microorganisms, and the copper dissolved in an acid solution is extracted from the base.
e) “In situ” (on-site) ore bioleaching: Ore deposits where minerals in a natural state or fractured due to former mining operations are leached directly where they are by irrigating the leaching solution over the surface in the presence of acidophilic microorganisms, and the copper dissolved in an acid solution is extracted from the base.
f) Ore bioleaching in tanks or stirred vessels: The bio-leaching process is carried out in a mechanically stirred reactor where the finely divided ore is mixed with the leaching solution, forming a slurry with up to 20% solid contents with presence of acidophilic microorganisms, and the copper is extracted dissolved in an acid solution.
g) Tailings dam bioleaching: tails that originate in the flotation process and have smaller quantities of metal in the ore, are gathered in dams from where they are extracted for leaching, whether in heaps or by stirring, in the presence of acidophilic microorganisms, and the copper is extracted dissolved in an acid solution.
h) Biomass: mass of live organisms produced in a specific area or volume.
i) DSM: “Deutsche Sammlung von Mikroorganismen und Zellkulturen GmbH” German Collection of Standard microorganism cultures.
j) Innoculum: pure or mixed bacterial culture that will act as active biological material during the bioleaching process.
k) Passivation: lowering of the leaching rate of an ore as a consequence of the accumulation of layers of sulfur and polisulphurs on its surface.
l) PLS: Aqueous solution generated in the bioleaching process, that contains the metallic ions leached from the ore. This solution constitutes the solvent extraction plant feed.
m) Raffinate: Aqueous solution depleted of copper as a result of the solvent extraction process.
n) Mixed energy source: substrate that allows the simultaneous growth of iron and sulfur-oxidizing microorganisms.
o) Mixed biomass: Mass of microorganisms capable of oxidizing reduced iron and copper compounds.

In order to achieve large-scale production of isolated microorganisms that are useful for sulfide metallic ore bioleaching, a process has been developed based on the use of bioreactors, with which it is possible to lower the costs of culture mediums used for the growth of these microorganisms, by using mixed energy sources.

This process consists in the use of a material containing pyrite to replace a part of the standard culture mediu in the way of a mixed energy source of two microorganisms of different types that grow together, that is to say Acidithiobacillus ferrooxidans and Acidithiobacillus thiooxidans.

This process also has advantages regarding the quantity of microorganisms and their adaptation to the solid phase, and advantages related to copper recovery and iron winning at +3 oxidation state as well.

According to the present invention, Acidithiobacillus ferrooxidans and Acidithiobacillus thiooxidans type microorganisms are cultured together along with other microorganisms, using a culture medium modified with pyrite which makes use of the presence and formation of species apt as energy sources: iron (oxidation state +2) and sulfur (oxidation state +2) respectively, and provides a series of advantages for the microorganism culture process.

Considering that part of the conventional culture medium has been replaced by a low cost material, it is obvious that this culture will be less expensive than the culture that uses a conventional medium. Furthermore, by culturing two microorganisms simultaneously, costs linked to premises, reactors, control systems, etc, which would otherwise have to be doubled, are also reduced.

Furthermore, joint culture using pyrite also makes it possible to obtain a higher concentration of microorganisms than would normally be obtained when the same microorganisms are cultured separately. This is of economic importance, a fact that can be assessed by the reduction of equipment needed to obtain a specific target concentration when new facilities are projected, or by a higher production capacity at facilities currently operating.

Based on studies carried out presented further on in the examples, it is possible to state that the association of microorganisms that contemplates isolated microorganisms mixed with microorganisms that are native in ores, grows normally in the modified medium with materials that contain it. This is progress regarding the state-of-the-art, because it lowers culture costs by reducing culture medium costs.

On the other hand, according to the reactions discussed above, a higher concentration of the Acidithiobacillus thiooxidans species or equivalently, a larger relative growth of the Acidithiobacillus thiooxidans species will naturally be produced. This may or not turn out to be advantageous depending on considerations regarding subsequent processes in which generated biomass is used. Nevertheless, if it is desired or necessary, it is possible to balance microorganism growth by incorporating Fe⁺² in the form of ferrous sulfate (FeSO₄.7H₂O).

As it has been pointed out, the invention is verified in practice by replacing part of the microorganism standard culture medium, with a material containing pyrite. The culture medium fraction replaced is the one that corresponds to the iron and sulfur species, and it may be replaced within a wide margin, for instance, in a culture medium modified according to the invention, from 1 to 20 g/L of pyrite (on a 100% basis) can be used.

On the other hand, and due to the fact that materials containing pyrite are mostly solids, an adaptation of the microorganisms to solid phase sulfur oxidizing is achieved. This adaptation is useful and is also technical progress, because when microorganisms are adapted to the solid phase, they will rapidly populate the materials placed in heaps, dumps, tailings dams or other “in situ” (on-site) operations in which they are used, lowering the time associated with their leaching.

Finally, and according to the reactions presented above, an enrichment of iron in the culture medium, at oxidation state +3, is produced. As it is known in the technique, the presence of Fe⁺³ favors secondary ore leaching, and for this reason it also represents an advantage over other processes.

### DESCRIPTION OF THE INVENTION

The process of this invention for the joint culture of an association of Acidithiobacillus thiooxidans type and Acidithiobacillus ferrooxidans type microorganisms, using pyrite (FeS₂) as an energy source, is defined according to the following operating stages and conditions:

a) preparing a culture medium for Acidithiobacillus thiooxidans type and Acidithiobacillus ferrooxidans type microorganisms, by replacing part of this culture with pyrite;

b) the Ph value of this medium is adjusted within a range of 1.5 to 2.5;

c) the culture medium is inoculated with a mixture of Acidithiobacillus thiooxidans type and Acidithiobacillus ferrooxidans type isolated microorganisms with or without native microorganisms;

d) the temperature is adjusted within a range of 25 to 35° C.;

e) an air current enriched with CO₂, with 0.20% to 0.80% CO₂, is driven through.

The part of the culture medium replaced in stage (a) is the part corresponding to reduced iron and sulfur compounds, such as ferrous sulfate and elemental sulfur.

In the process of the present invention, the culture medium with pyrite considers a quantity of pyrite of 1 to 20 grams per liter. The Acidithiobacillus thiooxidans and Acidithiobacillus ferrooxidans type microorganisms cultivated are isolated microorganisms, and the preferred Acidithiobacillus thiooxidans type microorganism is the Licanantay DSM 17318 while the preferred Acidithiobacillus ferrooxidans type microorganism is the Wenelen DSM 16786. The ratio of microorganism inoculum volume to culture medium volume ranges from 1:20 to 1:5.

### EXAMPLE 1

An experiment is carried out with the purpose of determining Wenelen DSM 16786 and Licantay DSM 17318 microorganism association growth kinetics and biomass performance, using a medium modified with the incorporation of pyrite concentrate (I), using the following protocol:

### Protocol

In order to achieve the proposed objective, a shaker-flask-type growth assay was carried out. The growth of the mixture of strains was carried out in 100 ml flasks on 25 ml of a culture medium supplemented with mixtures of two energy sources: pyrite concentrate (I)—whose characteristics are presented in Table 1—and ferrous sulfate, FeSO₄. The mixtures of energy sources used are detailed in Table 2. The culture medium nutrient composition was the following: 0.99 g (NH₄)₂SO₄/L, 0.128 g NaH₂PO₄.H₂O/L, 0.0525 g KH₂PO₄/L, 0.1 g MgSO₄.7H₂O/L, 0.021 g CaCl₂/L. Culture medium pH was adjusted to 1.8. The concentration of Wenelen DSM 16786 and Licanantay DSM 17318 strains in each flask was 2, 5·10⁷ cells/ml.

Flask incubation was carried out at 30° C. in an orbital shaker operated at 200 rpm.

Periodical follow-up of biomass concentrations in flasks was carried out by means of microscopic count in a Petroff-Hausser chamber, at six-day intervals.

TABLE 1
Pyrite concentrate (I) mineralogical composition

| Minerals | % Weight | % Vol. | % S | % Cu | % Fe | % As | % Mo | % Zn | % Pb |
|---|---|---|---|---|---|---|---|---|---|
| Chalcopyrite | 11.24 | 10.98 | 3.93 | 3.888 | 3.42 | | | | |
| Chalcosite | 10.41 | 7.50 | 2.09 | 8.315 | | | | | |
| Covellina | 5.57 | 4.97 | 1.87 | 3.705 | | | | | |
| Bornite | 7.74 | 6.23 | 1.98 | 4.902 | 0.86 | | | | |
| Cu G Tenantite | 0.17 | 0.15 | 0.04 | 0.088 | | 0.034 | | | |
| Enargite | 4.30 | 4.01 | 1.40 | 2.075 | | 0.816 | | | |
| Pyrite | 32.09 | 26.35 | 17.13 | | 14.95 | | | | |
| Molybdenite | 2.34 | 2.04 | 0.94 | | | | 1.40 | | |
| Galene | 0.13 | 0.52 | 0.02 | | | | | | 0.11 |
| Sphalerite | 4.13 | 4.23 | 1.36 | | | | | 2.77 | |
| Hematite | 0.09 | 0.07 | | | 0.07 | | | | |
| Limonite | 0.27 | 0.30 | | | 0.17 | | | | |
| Rutile | 0.15 | 0.15 | | | | | | | |
| Gangue | 21.38 | 32.50 | | | | | | | |
| Total | 100.00 | 100.00 | 30.77 | 22.972 | 19.47 | 0.850 | 1.40 | 2.77 | 0.11 |

TABLE 2
Mixture of energy sources used in Example 1 growth assays.

| | | Pyrite Concentrate (I) |
|---|---|---|
| Flask | FeSO₄•7H₂O [g/L] | pyrite[g/L] |
| 1 | 7.5 | 0 |
| 2 | 15.0 | 0 |
| 3 | 7.5 | 1 |
| 4 | 7.5 | 2 |
| 5 | 7.5 | 5 |
| 6 | 7.5 | 10 |

### EXAMPLE 1 RESULTS

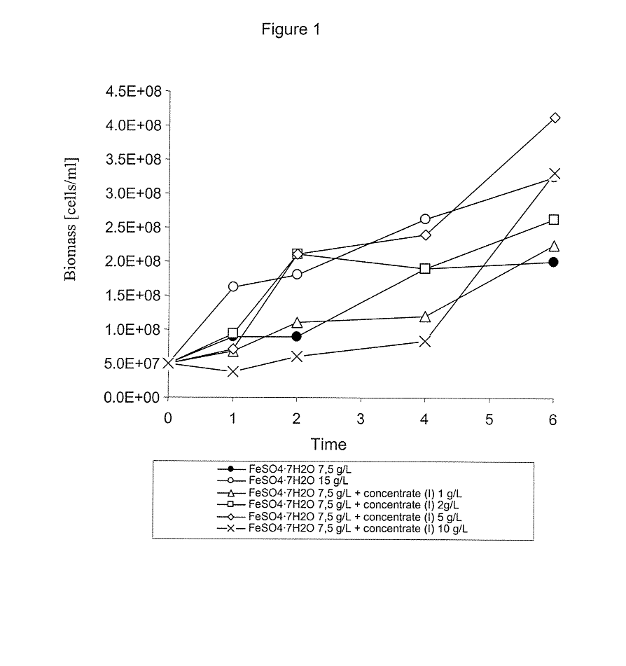

As it can be observed in FIG. 1, adding pyrite concentrate (I) at concentration levels of 2 and 5 g/1 makes it possible to increase the free biomass propagation rate and the final biomass title obtained in a medium with a 7.5 g/l initial ferrous sulfate concentration. Adding 10 g/L concentrate (I) makes only increasing the final title possible because of the delay in free biomass propagation probably due to the adsorption of cells on the solid surface. In the case of the 7.5 g/l ferrous sulfate +concentrate (I) 5 g/1 mixture, it is possible to obtain more free-biomass in 6 days than with a culture medium without concentrate (I) and with a 1.5 g/l ferrous sulfate concentration. In other words, it is clearly established that it is possible to replace part of the ferrous sulfate of the medium with pyrite concentrate (I).

### EXAMPLE 2

The following protocol is used to carry out an experiment with the purpose of determining growth kinetics and biomass performance of the Wenelen DSM 16786 and Licanantay DSM 17318 microorganism association using a medium modified with the incorporation of pyrite concentrate (II).

### Protocol

Bacterial growth was carried out in a 6 m³ reactor.

The culture medium used in microorganism propagation was prepared by suspending pyrite concentrate (II), whose characteristics are presented in Table 3, at 1.25% pulp density, in a nutrient solution composed of: 75 g FeSO₄/L, 0.99 g (NH₄)₂SO₄/L, 0.128 g NaH₂PO₄.H₂O/L, 0.0525 g KH₂PO₄/L, 0.1 g MgSO₄.7H₂O/L, 0.021 g CaCl₂/L. Culture medium pH was adjusted at 1.8.

TABLE 3
Pyrite concentrate (II) mineralogical composition

| Minerals | % Peso | % Vol. | % S | % Cu | % Fe | % Mo | % Zn |
|---|---|---|---|---|---|---|---|
| Chalcopyrite | 2.08 | 1.51 | 0.73 | 0.72 | 0.63 | | |
| Chalcosite | 0.53 | 0.29 | 0.11 | 0.43 | | | |
| Covellina | 0.62 | 0.41 | 0.21 | 0.41 | | | |
| Bornite | 1.37 | 0.82 | 0.35 | 0.86 | 0.15 | | |
| Pyrite | 19.28 | 11.76 | 10.30 | | 8.98 | | |
| Molibdenite | 0.94 | 0.61 | 0.38 | | | 0.57 | |
| Sphalerite | 0.05 | 0.04 | 0.02 | | | | 0.04 |
| Magnetite | 0.21 | 0.12 | | | 0.15 | | |
| Limonite | 0.20 | 0.16 | | | 0.13 | | |
| Rutile | 0.17 | 0.12 | | | | | |
| Gangue | 74.54 | 84.16 | | | | | |
| Total | 100.00 | 100.00 | 12.08 | 2.42 | 10.05 | 0.57 | 0.04 |

In order to start the culture, 5.400 L of culture medium were mixed with 600 L of bacterial inoculum carrying Wenelen DSM 16786 and Licanantay DSM 17318 microorganisms.

Air enriched with 0.5% of CO₂ was fed to the reactor to allow the growth of microorganisms in it. Reactor temperature was controlled at 30° C. The pH in the reactor was controlled by adding H₂SO₄.

The reactor was operated in a batch mode for 15 days. During the operation of the reactor, microorganism growth was monitored by means of microscopic count using a Petroff Hausser chamber.

### EXAMPLE 2 RESULTS

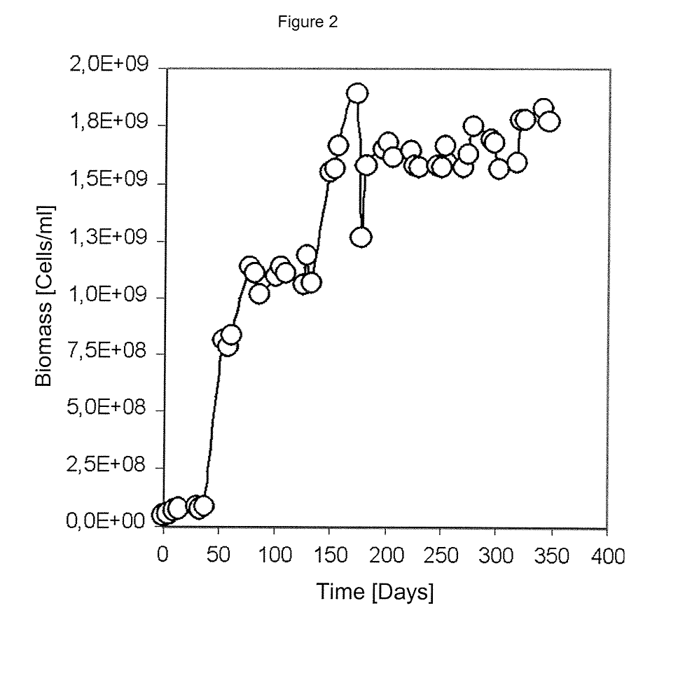

As it can be observed in FIG. 2, a rapid increase in microorganism concentration was produced in the culture medium modified with pyrite concentrate, and a maximum microorganism concentration of 1.7×10⁹ cells/ml was reached in 6 days. Based on data obtained during the exponential growth phase, it was possible to determine a specific 0.069 h⁻¹ growth rate.

### EXAMPLE 3

An experiment is carried out with the purpose of demonstrating that the Wenelen DSM 16786 and Licanantay DSM 17318 microorganism association can be effectively propagated in a continuous manner using a medium modified with the incorporation of pyrite concentrate (III), using the following protocol.

### Protocol

Bacterial growth was carried out in a 50 m³ reactor. The culture medium used in the propagation of the microorganisms was prepared by suspending pyrite concentrate (III) (at a 0.125% slurry density) in a nutrient solution composed of: 8 g FeSO₄/L, 0.99 g (NH₄)₂SO₄/L, 0.128 g NaH₂PO₄.H₂O/L, 0.0525 g KH₂PO₄/L, 0.1 g MgSO₄.7H₂O/L, 0.021 g CaCl₂/L. The pH of the culture medium was adjusted at 1.8.

In order to start the culture, 44 m³ of culture medium were mixed with 6 m³ of bacterial inoculum carrying Wenelen DSM 16786 and Licanantay DSM 17318 microorganisms.

Air enriched with 0.5% CO₂ was fed to the reactor to allow microorganism growth in it. The temperature in the reactor was controlled at 30° C. The pH in the reactor was controlled by adding H₂SO₄.

During the operation of the reactor, microorganism growth was monitored by means of microscopic count using a Petroff-Hausser chamber.

Characterization of microorganisms present in the reactor was carried out using the quantitative PCR technique (qPCR).

The reactor was operated in a batch mode for 7 days, after which its continuous operation was begun by feeding the culture medium of the prescribed composition at a 360 L/h rate.

During the reactor's continuous operation phase samples were taken in order to carry out their characterization with qPCR.

### EXAMPLE 3 RESULTS

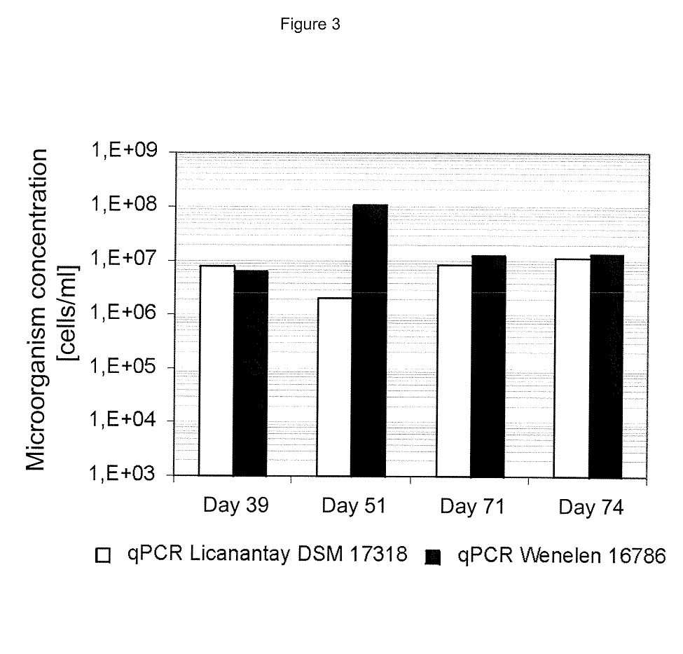

FIG. 3 shows that the continuous operation of a bioreactor, using a medium modified with the incorporation of pyrite concentrate, effectively permits the propagation of At. ferrooxidans and At. thiooxidans species

### ADVANTAGES OF THE INVENTION

In order to assess culture medium cost reduction as a result of the incorporation of pyrite concentrate, a 2000-ton heap is considered, irrigated with a 480 L/h flow; with continuous inoculation with a concentration of 1.3-10⁸ cells/mL.

The conditions indicated above determine a production requirement of 360 L/h of a microorganism culture, with a concentration of 1.3-10⁸ cells/ml. If a US $350 value per ton of ferrous sulfate with a concentration of 8 g/l is considered, the total substitution of this reagent by pyrite concentrate (I) would mean saving 8.830 dollars per year. Typical copper mining operations involve leaching over 2 million tons of ore per year (for example, Cerro Colorado operation in Chile), and for this reason, savings associated with the use of pyrite instead of ferrous sulfate and a source of sulfur separately, are over US $ 8 million a year.

1. Process for the joint culture of an association of Acidithiobacillus thiooxidans and Acidithiobacillus ferrooxidans type microorganisms; the process including:
a) preparing a culture medium for Acidithiobacillus thiooxidans type and Acidithiobacillus ferrooxidans type microorganisms replacing part of it with pyrite;
b) adjusting the pH value of this medium at 1.5 to 2.5;
c) inoculating the culture medium with a mixture of Acidithiobacillus thiooxidans type and Acidithiobacillus ferrooxidans type microorganisms cultured with or without other microorganisms;
d) adjusting the temperature to a level between 25 and 35° C.;
e) blowing an air current enriched with CO₂ with 0.20% to 0.80% CO₂ through.

2. Process according to claim 1, wherein the culture medium which is replaced is the one corresponding to reduced iron and sulfur compounds, such as ferrous sulfate and elemental sulfur.

3. Process according to claim 1, wherein the culture medium with pyrite considers a quantity of pyrite of 1 to 20 grams per liter.

4. Process according to claim 1, wherein the Acidithiobacillus thiooxidans and Acidithiobacillus ferrooxidans type microorganisms cultivated are isolated microorganisms.

5. Process according to claim 4, wherein the Acidithiobacillus thiooxidans type microorganism is Licanantay DSM 17318, and the Acidithiobacillus ferrooxidans type microorganism is Wenelen DSM 16786.

6. Process according to claim 1, wherein culture pH is 1.8.

7. Process according to claim 1, wherein culture temperature is controlled at 30° C.

8. Process according to claim 1, wherein air is enriched with 0.5% CO₂.

9. Process according to claim 1, wherein the ratio of microorganism inoculum to culture medium volume is 1:20 to 1:5.
